(19) United States
(12) Patent Application Publication
(10) Pub. No.: US 2007/0270348 A1
(43) Pub. Date: Nov. 22, 2007

(54) Compositions and Methods for Modulating Insulin-Activated Nitric Oxide Synthase

(76) Inventors: Nighat N. Kahn, Greenlawn, NY (US); Asru K. Sinha, Calcutta (IN)

Correspondence Address: LICATA & TYRRELL P.C., 66 E. MAIN STREET, MARLTON, NJ, 08053, US

(21) Appl. No.: 11/554,067

(22) Filed: Oct. 30, 2006

Related U.S. Application Data
(63) Continuation-in-part of application No. PCT/US05/14830, filed on Apr. 29, 2005.
(60) Provisional application No. 60/566,506, filed on Apr. 29, 2004.

(51) Int. Cl. A61K 38/08; A61P 3/10; A61P 35/00; C07K 7/06

(52) U.S. Cl. 514/15; 530/328

(57) ABSTRACT

Compositions and methods are provided for increasing nitric oxide synthesis in cells or tissues through use of a non-insulin polypeptide. The uses of the polypeptide include treatment of cancer, diabetes mellitus and hyperglycemia.

This application is a continuation-in-part of PCT/US2005/014830, filed Apr. 29, 2005, which claims benefit of priority to U.S. Provisional Patent Application Ser. No. 60/566,506, filed on Apr. 29, 2004, whose contents are incorporated herein by reference in their entireties.

### FIELD OF THE INVENTION

The present invention is related to polypeptides useful for promoting production of nitric oxide in body tissues and thus treating diseases wherein increased nitric oxide activity is beneficial, diseases that would include cancer and diabetes mellitus.

### BACKGROUND OF THE INVENTION

Nitric oxide is a chemical that has been implicated in many processes in the body, including regulation of blood pressure, defense against infection, function of the platelets and transmission of some types of nerve impulses. Nitric oxide has been implicated in neurotoxicity associated with stroke and neurodegenerative diseases, neuronal regulation of smooth muscle including peristalsis, and penile erections. Nitric oxide has been proposed to be a messenger molecule for its diversified effects in various physiologic and pathologic events (Ignarro (1990) Ann. Rev. Pharmacol. Toxicol. 30:535-560). Unlike typical neurotransmitters, nitric oxide is not stored in synaptic vesicles and does not act on membrane receptors.

Incubation of various tissues including heart, liver, kidney, muscle and intestine from mice; and erythrocytes of membrane fractions from humans, with physiologic concentrations of insulin has resulted in activation of a membrane-bound nitric oxide synthase (NOS). Activation of NOS and synthesis of nitric oxide were stimulated by the binding of insulin to specific receptors on the cell surface (Khan et al. (2000) Life Sci. 49:441-450). It was further demonstrated that a membrane bound form of nitric oxide synthase in human erythrocytes could be activated by insulin (Bhattacharya et al. (2001) Arch. Physiol. Biochem. 109:441-449). Insulin has been established to have an essential role in carbohydrate metabolism and is used to treat disorders of blood glucose metabolism, such as diabetes mellitus.

Diabetes mellitus (DM) is a risk factor for death worldwide aligned with other diseases such as cancer and a variety of cardiovascular disorders. In the United States the overall prevalence of DM has risen from 4.9% in 1990 to 6.5% in 1998, an increase of 33%. In addition to the approximately 15 million diagnosed cases of DM, more than five million additional Americans have DM that is undiagnosed. Current treatment of DM involves frequent monitoring and repeated subcutaneous or oral administration of agents that have a short duration of action.

DM is a chronic systemic disease characterized by disorders in metabolism of insulin, carbohydrate, fat and protein. DM is associated with hyperglycemia, and is classified as either insulin-dependent (IDDM type 1), which is related to absolute insulin deficiency caused by autoimmune illness that results in lack of insulin production because the body attacks its own insulin producing cells in the pancreas, or non-insulin-dependent DM (IDDM, type 2). Type 2 is further categorized as non-obese NIDDM (type 1, IDDM in evolution), obese NIDDM, or maturity-onset diabetes of the young (MODY).

Hyperglycemia is defined as persistently elevated fasting plasma glucose concentrations. Hyperglycemia is due to a systemic insulin deficiency, which can usually be corrected by subcutaneous injection of insulin. Insulin, a protein hormone derived from an animal pancreas, cannot be taken orally to control hyperglycemia because the hormone is completely and rapidly inactivated by the digestive juices.

As such, the need for an oral composition that is as effective as insulin in the control of hyperglycemia and DM has long been sought. One of the advantages of an oral alternative to insulin is the elimination of painful injections and possible complications of lipodystrophy, changes in the subcutaneous fat at the site of injection, or infections due to repeated insulin injections.

Compared to individuals without diabetes, diabetic patients are highly susceptible to complications of disease such as blindness, kidney disease and heart disease. With the use of insulin therapy, the acute or fatal symptoms of diabetes can be controlled, but the long-term complications reduce life expectancy. Insulin-dependent diabetes mellitus is caused by damage of insulin-producing pancreatic beta cells, which leads to a decrease in the amount of insulin and finally results in hyperglycemia. Hyperglycemia, or high blood glucose, usually develops slowly over several days because of insufficient production of insulin, inefficient use of insulin, or increased glucose production. As a result, glucose levels build in the blood and the body is unable to remove the excess glucose through the urine. In contrast, hypoglycemia or low blood glucose levels, occur when there is too much insulin in the blood, and insufficient glucose going to the brain and muscles prevents normal functioning of the brain and muscles.

WO 03/061679 describes a nutritional combination useful for treatment of abnormal sugar metabolism. The nutritional composition disclosed requires French lilac/Goat's rue or cinnamon in combination with American ginseng bitter melon, Gymnema sylvestre or garlic. In contrast, the present invention provides a novel protein that can control hyperglycemia in patients with DM.

U.S. Pat. No. 6,403,830 discloses novel compounds that selectively inhibit the inducible isoform of NOS over the constitutive isoforms of NOS and then the use of these compounds to treat diseases such as diabetes and cancer. However, this patent does not teach compounds that stimulate nitric oxide production, nor does it teach compounds that affect activity of forms of NOS other than the inducible form.

Nitric oxide has also been shown to possess a wide range of anti-neoplastic properties. The anti-neoplastic properties of the molecule include induction of apoptosis and differentiation in cancer cells (Farias-Eisner et al. (1994) Proc. Natl. Acad. Sci. 91:9407-9411; Jun et al. (1996) Kor. J. Exper. Mol. Med. 28:101-108) and in the production of anti-invasive anti-tumor maspin in epithelial cells in breast cancer (Moshin et al. (2003) J. Pathol. 199:432-435; Zou et al. (1994) Science 263:526-529). It has also been reported that plasma nitric oxide levels are decreased in various neoplastic conditions when compared to normal conditions (Sinha et al. (2002) J. Can. Res. Clin. Oncol. 128:659-668). The decrease in plasma nitric oxide levels in cancer patients was found to be related to the appearance of an antibody against insulin-activated NOS, a constitutive and product-regulated form of membrane-bound NOS in various cells, including human erythrocytes (Kahn et al. (2000) Life. Sci. 49:441-450). Restoration of the impaired insulin-activated NOS activity resulted in modification of clinical outcomes in different cancers, including regression of solid tumors (Sinha et al. (2002) J. Can. Res. Clin. Oncol. 128:659-668).

### SUMMARY OF THE INVENTION

The present invention relates to a non-insulin polypeptide for increasing nitric oxide levels in cells or tissues. The polypeptide of the invention contains the amino acid sequence Glu-Gly-Leu-Tyr-Ala-Gly-Gln-Ser-Leu (SEQ ID NO:15) and is capable of increasing the synthesis of nitric oxide in cells or tissues. In one embodiment, the polypeptide is formulated as a pharmaceutical composition by combining the polypeptide with a pharmaceutically acceptable carrier.

The present invention also provides methods for increasing nitric oxide synthesis in cells or tissues of animal, including humans; methods for controlling hyperglycemia; methods for preventing hyperglycemia; methods for increasing insulin production; and methods for killing cancer cells using the non-insulin polypeptide of the invention which increases the synthesis of nitric oxide in cells or tissues.

### DETAILED DESCRIPTION OF THE INVENTION

Nitric oxide (NO) is a biologic messenger molecule. Nitric oxide is believed to be an endothelium derived relaxing factor. It is believed that nitric oxide production is an obligatory step in the elicitation of the hypoglycemic effect of insulin. It is now known that proteins other than insulin can stimulate nitric oxide synthesis and consequently produce insulin-like effects of hypoglycemic in the system. Nitric oxide also possesses a wide range of anti-neoplastic properties and an insulin-activated NOS has been shown to be activated in cell membranes of a variety of cells, with cancerous tumors producing an antibody that blocks insulin-activated NOS activity. Therefore, the present invention includes compositions and methods for treating diseases associated with nitric oxide activity, specifically diseases where an increase in nitric oxide activity is desired, including but not limited to diabetes mellitus and cancer.

Applications to Treatment of Cancer

It is believed that neoplastic cells elicit the aid of an antibody in the system capable of blocking the production of nitric oxide through activation of insulin-activated NOS (IANOS) by insulin. Unlike normal cells, cancer cells do not produce nitric oxide when treated with insulin. Further, cancer cells do not need insulin for the stimulation of carbohydrate metabolism. Nitric oxide only stimulates carbohydrate metabolism in normal cells. However, nitric oxide acts as a potent tumoricide in cancerous cells. The antibody against IANOS plays a crucial role in the pathophysiology of cancer through blocking of IANOS. The antibody against IANOS is the light chain protein of IgG. This antibody occurs in both humans and animals with neoplastic diseases and cancers.

The present invention provides a polypeptide that can be used to treat cancer in an animal, including humans, by stimulating the production of nitric oxide in tissues or cells of the body of the animal. Therefore, the present invention is also a method of stimulating production of nitric oxide in cells or tissues of the body, wherein the stimulation of nitric oxide production is also a method for treating a disease, including but not limited to cancer. Types of cancer that can be treated would include, but not be limited to, non-hodgkin's lymphoma, hodgkin's lymphoma, acute lymphocytic leukemia, acute myeloid leukemia, multiple myeloma, renal cell carcinoma, brain, breast with mastectomy, breast without mastectomy, lung (non-small cell), lung, esophagus, liver, gall bladder, colon, rectal, uterine, cervical, ovarian, prostrate, tongue, pyriform fossa, mandible, pancreatic or bone cancer.

Applications to Treatment of Diabetes Mellitis

Nitric oxide is a biologic messenger molecule and also an endothelium-deriving relaxing factor. Nitric oxide production is an obligatory step in the elicitation of the hypoglycemic effect of insulin. It is now known that proteins other than insulin can stimulate nitric oxide synthesis and consequently produce insulin-like effects, including hypoglycemia.

The present invention provides a polypeptide that can be used to treat diabetes mellitis in an animal, including humans, by stimulating the production of nitric oxide in tissues or cells of the body of the animal. This polypeptide is mimicking the effect of insulin in the body. Therefore, the present invention is also a method of stimulating production of nitric oxide in cells or tissues of the body, wherein the stimulation of nitric oxide production is also a method for producing hypoglycemia and treating a disease including, but not limited to, diabetes mellitis.

Identification/Purification of Nitric Oxide Stimulating Agent

In the present invention, aqueous extracts of various fruits and vegetables were screened for their efficacy to stimulate nitric oxide synthesis from l-arginine in vitro using a human erythrocyte suspension and it was discovered that an aqueous extract of garlic contained a potent simulator of nitric oxide synthesis. The active agent in the extract, was determined to be a polypeptide, which was purified to homogeneity and is referred to hereinafter as allimin. The procedure for purification of allimin encompassed extraction of the garlic with benzene followed by chromatography on a SEPHADEX® G 50 column, which provided for a 1342-fold purification of allimin over the starting material (Table 1). Electrophoresis of the purified allimin on SDS-polyacrylamide gel under non-reducing and reducing conditions demonstrated that the purified protein was a single chain molecule with a molecular weight of 4 kD.

TABLE 1

| | | Specific | |
|---|---|---|---|
| | Total | Activity | Fold |
| Step | Protein (mg) | (nmol NO/mg/h) | Purification |
| crude extract | 1000 | 0.42 | |
| benzene cut 1 | 500 | 0.77 | 1.83 |
| benzene cut 2 | 250 | 2.73 | 6.5 |
| benzene cut 3 | 135 | 3.38 | 8 |
| SEPHADEX ® G-50 | 1.788 | 563.75 | 1342 |

The purified allimin amino acid sequence was identified as Xaa-Met-Ile-Pro-Thr-Asn-Gly-Glu-Gly-Leu-Tyr-Ala-Gly-Gln-Ser-Leu-Asp-Val-Glu-Gln-Tyr-Lys-Phe-Ile-Met-Arg-Pro-Asp-Asp-Asn-Leu-Val-Xaa-Tyr-Xaa (SEQ ID NO:1), wherein Xaa were unidentified amino acid residues. The purified allimin polypeptide exhibited a hypoglycemic effect as well as other insulin mimetic properties, and was also shown to have anti-neoplastic properties. Oral administration of allimin was found to be capable of controlling elevated blood glucose levels in alloxan-induced diabetic mice.

While there was limited overall homology between insulin and allimin, it was unexpectedly found that there are domains which share cross-species homology between the human insulin precursor (GENBANK Accession No. P01308; SEQ ID NO:2), a 110 amino acid residue produced by the pancreas prior to the production of insulin, and allimin (SEQ ID NO:1), a 35 amino acid residue polypeptide (Table 2).

TABLE 2

| | | |
|---|---|---|
| | SEQ ID | % Identity/ |
| Sequence Alignment | NO: | % Positive |

| | | | | | |
|---|---|---|---|---|---|
| Insulin: | 59 | EDLQVGQ | 65 | 3 | 57%/57% |
| | | E L GQ | | | |
| Allimin: | 7 | EGLYAGQ | 13 | 4 | |
| Insulin: | 79 | PLALEGSLQKRGI-VEQ | 94 | 5 | 35%/46% |
| | | P EG + + VEQ | | | |
| Allimin: | 3 | PTNGEGLYAGQSLDVEQ | 19 | 6 | |
| Insulin: | 69 | GGGPGAGSLQPLALE | 83 | 7 | 46%/52% |
| | | G G AG Q L +E | | | |
| Allimin: | 6 | GEGLYAG--QSLDVE | 18 | 8 | |

Further, allimin exhibited significant sequence identity with a portion of the 181 amino acid residue mannose-specific lectin from garlic (A. sativum) (amino acid residues 31-65 of GENBANK Accession No. AAB64237) and sequence identity with portions of other mannose-specific lectins found in a variety of Allium species including A. porrum (leek, amino acid residues 31-65 of GENBANK Accession No. AAC37361), A. cepa (onion, amino acid residues 16-50 of GENBANK Accession No. AAC37359), A. ascalonicum (shallot, amino acid residues 27-61 of GENBANK Accession No. AAC37360), A. ursinum (wild garlic, amino acid residues 28-62 of GENBANK Accession No. AAA16280), and A. triquetrum (threecorner leek, amino acid residues 29-63 of GENBANK Accession No. ABA00714). See Table 3.

TABLE 3

| | | |
|---|---|---|
| | | SEQ |
| | | ID |
| Source | Sequence | NO: |

| | | |
|---|---|---|
| Allimin | XMIPTNGEGLYAGQSLDVEQYKFIMRPDDNLVXYX | 1 |
| A. sativum | RNLLTNGEGLYAGQSLDVEQYKFIMQDDCNLVLYE | 9 |
| A. porrum | RNLLTNGEGLYAGQSLDVEQYKFIMQDDCNLVLYE | 10 |
| A. cepa | RNVLVNNEGLYAGQSLVVEQYTFIMQDDCNLVLYE | 11 |
| A. ascalon- | RNVLVNNEGLYAGQSLVEEQYTFIMQDDCNLVLYE | 12 |
| icum | | |
| A. ursinum | RNLLGNGEGLYAGQSLEEGPYKLIMQEDCNLVLYE | 13 |
| A. trique- | RNILLNGEGLYAGQSLEEGPYRLAMQDDCNLVLYD | 14 |
| trum | | |
| | * ********* * * * *** * | |
| | EGLYAGQSL | 15 |

Other plants further believed to contain allimin include: Tulipa species; Narcissus hybrid cultivar lectin 5/19, Clivia miniata, Galanthus nivalis or common snowdrop, Hyacinthoides hispanica, Polygonatum multiflorum bacteria, Vibrio cholerae (outer membrane), and Listera ovata. The polypeptide described in the present invention has similarities with polypeptides found in the above listed vegetation and plants. The cross-species similarity is categorized as mannose-specific and lectin precursor binding.

It is contemplated that the mannose-specific lectin is the precursor of allimin, a lectin capable of binding to the cell-surface glycoproteins in the digestive tract. It is further contemplated that the binding of allimin to the cell-surface glycoproteins confers resistance to the enzymatic degradation of the protein in the gastrointestinal tract or facilitates uptake into circulation thereby leading to the increase in plasma nitric oxide level resulting in the systemic control of hyperglycemia.

In accordance with the present invention, an allimin polypeptide can be purified from an Allium species as disclosed herein, recombinantly produced using commercially available expression systems, or synthetically produced using conventional methods. The amino acid sequence of an allimin polypeptide of the present invention can be based upon the amino acid sequence of allimin itself or its Allium orthologs identified in A. sativum, A. porrum, A. cepa, A. ascalonicum, A. ursinum, and A. triquetrum. An allimin polypeptide of the invention can be purified in its mature form or obtained by cleavage of a mannose-specific lectin from an Allium species. In some embodiments, the allimin polypeptide of the invention is 5 to 50 amino acid residues in length, 10 to 40 amino acid residues in length, or 15 to 35 amino acid residues in length. In other embodiments, the allimin polypeptide is less than 50 amino acid residues, less than 45 amino acid residues, or less than 40 amino acid residues in length. While some embodiments embrace an allimin polypeptide having the amino acid sequence Glu-Gly-Leu-Tyr-Ala-Gly-Gln-Ser-Leu (SEQ ID NO:15) other embodiments embrace an allimin polypeptide having an amino acid sequence set forth in SEQ ID NO:1, SEQ ID NO:9, SEQ ID NO:10, SEQ ID NO:11, SEQ ID NO:12, SEQ ID NO:13, or SEQ ID NO:14.

Experiments Demonstrating Anti-Diabetic Activity

No isolated protein, other than insulin itself, has been reported to control hyperglycemia in diabetic animals either by injection or by oral administration. However, the hypoglycemic effect of the garlic-derived protein allimin was found to be more effective than the hypoglycemic effect of insulin. A single bolus administration of allimin by mouth was capable of maintaining euglycemia for four to five consecutive days in diabetic animals. In contrast, single intramuscular injection of insulin only resulted in the control of hyperglycemia in these diabetic animals for less than 24 hours. Oral administration of insulin produced no effect on the elevated plasma glucose level in these diabetic animals. The hypoglycemic effect of allimin can be extended to diabetes mellitus in man, making the protein useful to control hyperglycemia by oral administration of allimin to humans with this condition.

Although allimin is structurally unrelated to insulin, the hypoglycemic effect of both proteins is believed to be mechanically related. The hypoglycemic effect of insulin is associated with the ability of the hormone to stimulate the synthesis of nitric oxide due to the interaction of the hormone with the cell surface insulin receptor. The stimulation of nitric oxide synthesis by insulin is an obligatory step for the hypoglycemic effect of the protein. Nitric oxide also has a hypoglycemic effect in insulin-sensitive tissues. Garlic was not chosen at random for the isolation of insulin mimicking protein, but rather allimin was isolated and identified by screening different proteins from various sources for the ability to stimulate nitric oxide synthesis in human erythrocytes. Thus, it has been discovered that allimin modulates insulin production and promotes insulin production in a subject.

Furthermore, the result disclosed herein show that insulin, a pancreatic protein, is not unique in its ability to control hyperglycemia in diabetes mellitus, and that many other proteins like allimin, found in both the plant and animal kingdoms, exist which would mimic the effect of insulin. The hypoglycemic effect of allimin demonstrates that nitric oxide is capable of regulation of insulin, and that agents capable of increasing systemic nitric oxide levels reduce blood glucose levels in a subject.

Furthermore, the addition of Alloxan, (NG-nitro L-arginine methyl ester, hereinafter referred to as “NAME”), a potent inhibitor of both nitric oxide synthesis and transduction of insulin effect in the stimulation of carbohydrate metabolism in the hormone responsive tissue, to reactions containing allimin completely inhibited the allimin-stimulated nitric oxide synthesis in erythrocyte suspensions. Injection of 10 μM NAME in 0.9% NaCl to a diabetic mice 30 minutes prior to the oral administration of allimin also blocked the expected hypoglycemic effect of the garlic protein. These results are similar to the blockade of the hypoglycemic effect of insulin due to the in vitro inhibition of the synthesis of nitric oxide.

The insulin-mimetic effect of allimin is not only effective to the control hyperglycemia in diabetic animals and humans, it also mimics the effects of insulin in relation to glucose transport, glucose oxidation, stimulation of tyrosine kinase and PI₃ kinase.

Addition of NAME to allimin reaction mixtures also inhibited the allimin-induced nitric oxide synthesis. The stimulation of carbohydrate metabolism in mouse epitrochlearis muscle and membrane is shown in Table 4. The materials used were Alloxan, Protein A antibodies to PI₃ kinase and phosphotyrosine kinase, glu⁸⁰-Tyr⁴⁰ Copolymer 2 deoxy D-¹⁴C glucose (210 mCi/mmol) and [U-¹⁴C]-D-glucose (310 mCi/mmol) (obtained from Sigma Chemical Co., St. Louis, Mo.), [α-³²P]-ATP, specific activity 10 mCi/mmol (obtained from Amersham Corp). Insulin (trade name HUMULIN was obtained from Eli Lilly, Indianapolis, Ind.). The stimulation of membrane tyrosine kinase and PI₃ kinase by allimin indicated that, as in the case of insulin, the activation of these enzymes is believed to be a prerequisite to the synthesis of nitric oxide by the protein allimin.

TABLE 4

| | | | | Tyrosine | |
|---|---|---|---|---|---|
| | | Glucose | Glucose | kinase | PI3 kinase |
| Tissue | Addition | transport* | Oxidation* | activation^{#} | activation^{#} |
| Muscle | None | 63 ± 5 | 196 ± 75 | | |
| Muscle | Insulin | 75 ± 4 | 278 ± 80 | | |
| | (200 μU/ | | | | |
| | ml) | | | | |
| Muscle | Allimin | 82 ± 6 | 306 ± 82 | | |
| | (50 μg/ml) | | | | |
| Muscle | Allimin + | 57 ± 3 | 170 ± 6 | | |
| | NAME | | | | |
| | (10 μg/ml) | | | | |
| Membrane | None | | | 100 ± 15 | 100 ± 4 |
| Membrane | Insulin | | | 210 ± 18 | 155 ± 10 |
| | (200 μU/ | | | | |
| | ml) | | | | |
| Membrane | Insulin + | | | 215 ± 10 | 162 ± 6 |
| | NAME | | | | |
| Membrane | Allimin | | | 215 ± 10 | 187 ± 12 |
| | (50 μg/ml) | | | | |

*Data presented as nmol/g protein/minute (mean ± SD).
^{#}Data presented as % activation (mean ± SD).

It was further discovered that basal plasma nitric oxide levels in non-diabetic mice was seven-fold higher than that in the diabetic animals. Allimin (75 micro grams/kg body weight) was orally administered in a single bolus dose to alloxan-induced diabetic mice (n=10) group of animals received insulin (200 micro units/25 gm body weight) in their hind leg. Allimin can be administered in single or multiple doses. Allimin can be administered orally, topically, intranasally, via dermal patches or through any other suitable method of administration routinely used by one of skill in this field. Based upon the ease of administration, oral application may be the most favorable mode of administering allimin to a subject. Suitable subjects include humans, mice, cats, dogs, horses, pigs, or other diabetic or insulin-dependant animals. One oral dose has been found to be an amount effective at maintaining glucose at normal levels for a period of up to four or five days. Allimin can be routinely administered every 4 to 5 days or as necessary based upon indications of blood glucose levels rising above the normal post-prandial levels.

Although oral feeding of equal amounts of allimin resulted in greater increases in plasma nitric oxide levels in the non-diabetic mice when compared with that in diabetic mice, it was determined that the maintenance of plasma nitric oxide levels of about 0.4 nmolar was adequate to control the hyperglycemia in these animals. It was also discovered that about five days was required before the allimin-induced increase of nitric oxide level returned to basal diabetic level (0.18 nmol/ml) in these diabetic animals. During this time, the hyperglycemia was controlled during the period when the plasma nitric oxide level remained in the ranges of 0.4 nmol/ml. Thus, allimin provides a means for treating diabetes or hyperglycemia with minimal dosages as compared to commercially available insulin which is injected one or more times daily. In the case of alloxan-induced diabetes mellitus in animal models, including humans, the plasma nitric oxide level was markedly decreased when compared to that in non-diabetic volunteers. These results indicated an impaired nitric oxide homeostasis in diabetes mellitus in general.

Given its sequence identity with mannose-specific lectins, it is contemplated that allimin binds to the cell surface glycoproteins in the digestive tract. It is believed that the binding of allimin to the cell-surface glycoproteins confers resistance to the enzymatic degradation of the protein in the gastrointestinal tract or alternatively assists the compound in entering circulation, leading to an increase in plasma nitric oxide levels and consequently leading to the systemic control of hyperglycemia.

The polypeptide of the present invention controls hyperglycemia via increased synthesis of nitric oxide upon contact with cell surface insulin receptors. In one embodiment the protein is allimin. The protein identified as allimin stimulates nitric oxide synthesis in human erythrocytes. Since the hypoglycemic effect of insulin is mediated through the increase of plasma nitric oxide levels, the effect of allimin on plasma nitric oxide levels and on hyperglycemia in alloxan-induced diabetic mice was determined. It was established that while the plasma nitric oxide levels in non-diabetic mice was 0.75±0.05 nmol/ml, the plasma nitric oxide levels in the diabetic animal was markedly decreased to 0.1±0.02 nmol/ml. After single oral administration of allimin (75 micrograms/kg of body weight) in 0.9% NaCl to the diabetic mice, the plasma nitric oxide levels increased to 0.45±0.05 nmol/ml within 24 hours and continued to increase thereafter, to a maximum of 0.8±0.1 nmol/ml on the third day. On the fourth day and onward, the plasma nitric oxide levels gradually decreased and on the sixth day the plasma nitric oxide level returned to the pretreatment levels in the diabetic mice. In non-diabetic mice, the oral feeding of allimin produced an increase in the plasma nitric oxide levels, which resulted in a maximum increase on the third day and thereafter, the plasma nitric oxide levels decreased to the pretreatment levels on the fourth day.

Plasma glucose levels were continuously monitored to determine whether the increased plasma nitric oxide level achieved through the oral administration of allimin would result in a decrease of elevated plasma glucose level in diabetic mice. It was established that the initial plasma glucose level, which was 470±20 mg/dl before the administration of allimin, decreased to 120±10 mg/dl (p=0.05, n=10) within the next twelve hours and remained within 120-130 mg/dl for the next four days. On the fifth day, the plasma glucose level began to increase and on the sixth day the plasma glucose level increased to above 350 mg/dl. Injection of diabetic mice with 10 μm NAME, thirty minutes prior to the oral feeding of allimin, resulted in the blockade of the hypoglycemic effect of the allimin polypeptide.

In another set of experiments wherein alloxan-induced diabetic mice received allimin by injection (200 μunits/25 g) in their hind leg muscle rather than by oral administration, it was shown that the injection of insulin promptly reduced the elevated blood glucose level to 110±20 g/dl similar to that of allimin. However, unlike allimin, the reduction of blood glucose level achieved through the injection of insulin again increased to 350±20 mg/dl within the next 24 hours. The oral administration of insulin to these diabetic mice produced no effect on the reduction of blood glucose level. The insulin mimetic effects of allimin were also demonstrated in glucose transport and oxidation and in the activation of the membrane receptor tyrosine kinase and PI₃ kinase.

The present invention provides a polypeptide for controlling or reducing blood glucose levels by increasing nitric oxide production in an animal thereby controlling or inhibiting the occurrence of hyperglycemia.

A method for identifying proteins useful to control hyperglycemia in a subject is also provided. The method encompasses mixing a protein suspected of stimulating nitric oxide synthesis from l-arginine in vitro with a suspension of human erythrocyte and measuring the efficacy of the protein to stimulate nitric oxide synthesis, wherein proteins which are efficient in the stimulation of nitric oxide synthesis are identified as useful to control hyperglycemia.

In one embodiment of the present invention, a garlic extract was treated three times with benzene and the aqueous phase containing allimin was subjected to chromatography on a SEPHADEX® G50 column. Fractions referred to as 86-93 showed the highest stimulation of nitric oxide synthesis from l-arginine in human erythrocyte preparations. Fractions 86-93 were eluted from the column in a single peak. As summarized in Table 1, the combined treatment of the garlic extract with benzene and SEPHADEX® G50 provided 1342-fold purification of allimin over the starting material. The final preparation had no odor, taste or color.

Electrophoresis of the purified allimin on SDS-polyacrylamide under alkaline conditions demonstrated that the purified protein was homogeneous in nature. The molecular weight of allimin was determined to be 4 kD. When the purified allimin was reduced using 1 mM dithiothreitol and electrophoresed in SDS-polyacrylamide gel under reducing conditions, the electrophoretic movement of the reduced protein remained unchanged, indicating that allimin was a single chain protein. The matrix-assisted laser desorption time of flight mass spectrometry and protein sequence analysis demonstrated that allimin was a protein with a molecular weight of 4 kD and is set forth herein as SEQ ID NO:1.

Stimulation of nitric oxide synthesis from l-arginine by allimin in human erythrocytes was shown by incubation of a human erythrocyte suspension with different concentrations of purified allimin. This analysis demonstrated that nitric oxide synthesis increased in the presence of increasing amounts of allimin. At a concentration of 8 nM allimin, the synthesis of nitric oxide was maximally stimulated. Addition of 10 μM NAME to the reaction mixture resulted in the complete inhibition of nitric oxide synthesis.

The effect of oral administration of allimin on the plasma nitric oxide level in diabetic and non-diabetic mice was determined using alloxan-induced diabetic mice as compared to normal non-diabetic mice. It was shown that the plasma nitric oxide level in the diabetic mice was markedly reduced compared to non-diabetic controls. Allimin was orally administered (75 μg/kg body weight) to non-diabetic and alloxan-induced diabetic mice. The plasma nitric oxide level was measured on different days. Results were obtained from 10 mice in each group in triplicate. While the plasma nitric oxide level in the diabetic mice was 0.1±0.02 nmol/ml, the nitric oxide level in the non-diabetic mice was 0.75±0.05 nmol/ml (p<0.05). Upon oral administration of allimin (75 micrograms/kg body weight), the plasma nitric oxide level was increased to 0.45±0.05 nmol/ml within 24 hours and continued to increase thereafter, to a maximum of 0.8±0.1 nmol/ml on the third day. On the fourth day, the plasma nitric oxide level began to gradually decrease. On the sixth day, the plasma nitric oxide level returned to the pretreatment level in the diabetic mice. In non-diabetic mice, the oral feeding of allimin also resulted in an increase in plasma nitric oxide levels which showed a maximum increase by the third day, and decreased to the pretreatment level on the fourth day.

Oral administration of allimin as a one time bolus dose resulted in persistent elevation of plasma nitric oxide in diabetic mice for four to five days. To determine whether the increased plasma nitric oxide level achieved through the oral administration of allimin would result in the decrease of elevated plasma glucose levels in diabetic mice, the plasma glucose level was continuously monitored. It was found that the initial plasma glucose level which was 470±20 mg/dl before the administration of allimin decreased to 120±10 mg/dl within the next 12 hours and remained within 120-130 mg/dl for the next four days. On the fifth day after the oral administration of allimin the plasma glucose level began to increase and on the sixth day the plasma glucose level increased above 350 mg/dl. In a further study, diabetic mice were injected with insulin (200 μunits/25 g) in their hind leg muscle instead of oral administration of allimin. It was determined that the injection of insulin promptly reduced the elevated blood glucose level to 110±20g/dl, similar to that in the case of allimin. However, unlike allimin, the reduction of blood glucose level achieved through the injection of insulin was temporary and increased to 350±20 mg/dl within the next 24 hours.

Oral administration of insulin to these diabetic mice produced no effect on the reduction of blood glucose level. When non-diabetic mice received similar oral administration of allimin (75 ug/kg body weight) no overt hypoglycemia was found to have developed in the non-diabetic mice during this period, as the blood glucose levels remained in the ranges of 80-110 mg/dl. It should however be noted that food and water were freely available to both diabetic and non-diabetic mice during this period.

Insulin mimetic effects of allimin were observed in relation to glucose transport, glucose oxidation, insulin receptor tyrosine kinase and PI₃ kinase activation. The in vitro effect of allimin on glucose transport and glucose oxidation was compared with that of insulin in mice epitrochlearis muscle which is reported to be a model target tissue for insulin action. Incubation of the epitrochlearis muscle with either 200 μunits/ml insulin or 50 μg/ml allimin resulted in the stimulation of glucose transport and glucose oxidation to similar ranges when compared with the control. Addition of NAME to the incubation mixture inhibited the insulin-mimicking effect of allimin on both glucose transport and glucose oxidation. In another study, membranes from epitrochlearis muscle were prepared and the effect of allimin on the activation of the membrane tyrosine kinase and PI3 kinase were determined by incubating the membrane preparation with either insulin or allimin. It was established that the activation of the membrane tyrosine kinase and PI₃ kinase by 200 μunits of insulin/ml were comparable to that obtained by using 50 μgrams of allimin/ml under otherwise identical conditions.

Anti-Neoplastic Effects of Allimin

Using a well-established mouse model for anti-neoplastic drug testing (Hartveit et al. (1970) Acta. Pathol. Microbiol. Scand. 78:516-524), the effects of oral administration of allimin on the production of nitric oxide in mouse erythrocytes was examined. Blood was drawn from the tail vein of Swiss albino mice (2 months of age) and was anti-coagulated by adding sodium citrate to the blood sample. The erythrocyte suspension was prepared according to standard methods (Ray et al. (1986) Biochim. Biophys. Acta. 856:421-427). Stimulation of nitric oxide synthesis was determined using 0.1 ml portions of different fractions from a SEPHADEX® G50 column and adding it to the mouse erythrocyte suspensions. The formation of nitric oxide in the reaction mixture was quantitated by the conversion of oxyhemoglobin to methemaglobin (Jia et al. (1996) Nature 380:221-226) and verified by chemiluminescence techniques (Sinha et al. (1999) Life Sci. 265:2687-2696). In addition to normal or control mice, ascitic carcinoma was induced in Swiss albino mice by injecting approximately 10⁶ Ehrlich's ascitic carcinoma cells (EAC cells) into the peritoneal cavity of mice (Hartveit et al. (1970) Acta. Pathol. Microbiol. Scand. 78:516-524). Animals were divided into two groups, one group receiving laboratory chow alone and the other receiving allimin (75 micrograms/kg body weight/day) along with laboratory chow. Animals in each group were given the respective diets for 10 days before both groups were injected with EAC cells. Dietary treatment then continued during ascites development such that an increase of an EAC cell count to 5×10⁶ cells/ml was achieved.

Nitric oxide synthesis levels were determined at different days after oral administration of allimin. Synthesis of nitric oxide decreased steadily in mice that did not receive allimin. In contrast, the synthesis of nitric oxide in erythrocytes was restored to normal levels at day 20 in mice that received allimin daily. Twenty days after injection of mice with EAC cells, nitric oxide production in erythrocytes was reduced by 86.72%, while mice receiving EAC cells and dietary allimin exhibited a significantly different reduction (only 12.48%; Table 5). All mice not receiving allimin succumbed to the malignancy by day 20. When the experiments were repeated with allimin fed 10 days after the carcinogenic insult, the nitric oxide production from the erythrocytes was restored to 92.78% and 4.02% in mice treated with and without allimin, respectively.

TABLE 5

| Day | No Treatment* | Allimin Treated* |
|---|---|---|
| 1 | 0.557 ± 0.02 | 0.569 ± 0.02 |
| 5 | 0.275 ± 0.02 | 0.392 ± 0.01 |
| 10 | 0.154 ± 0.01 | 0.408 ± 0.02 |
| 15 | 0.110 ± 0.01 | 0.440 ± 0.01 |
| 20 | 0.074 ± 0.01 | 0.498 ± 0.02 |
| 25 | — | 0.532 ± 0.02 |
| 30 | — | 0.528 ± 0.03 |

*nmol NO produced/10⁸ cells/hour

When survival of mice with Ehrlich's carcinoma ascites was considered, allimin was shown to have significant effects on survival. As discussed above, control mice not treated with allimin succumbed to the malignancy by day 20. In contrast, animals that received allimin treatment on a daily basis survived the EAC carcinogenic insult at 20 days. Ten percent of the allimin treated mice survived for at least 40 days. This was a statistically significant difference in survival (p<0.01). It was also found that in those mice receiving EAC injection and oral allimin, weight increased from 20.57±0.78 g to 38.68±1.34 g (p<0.01) due to peritoneal fluid accumulation. In the case of control mice (no allimin), body weight increased, again due to fluid accumulation, but the increase was greater (21.69±0.73 g to 55.76±1.69 g, p<0.01) after 21 days. Only 1-2% of control mice, which received only the injection of EAC cells, survived more than 22 days.

Experiments were performed to determine the effects of allimin on the viability of EAC cells, a measure of the anti-neoplastic activity of allimin. Total EAC count (dead+viable cells) in peritoneal fluid was determined after 14 days of injection of EAC in the allimin treated and the untreated mice. It was found that the total EAC count in peritoneal fluid was 7×10⁶ cells/ml in both cases. However, it was also found that the dead EAC count was 2×10⁵ cells/ml in the allimin-treated mice while the dead cell count in the untreated mice was only 3×10⁴ cells/ml. It was noted that 25% of the viable EAC in the fluid of allimin-treated mice were twice as large (size) as compared to those in the untreated mice. This increase in cell size is indicative of the onset of death and the loss of the ability to regulate osmotic pressure (Ballas (1999) Merck Manual, Merck Research Laboratories, Whitehouse Station, N.J., pp. 1002-1023). The cellular basis for the decreased viability of the carcinoma cells in the allimin-treated mice was tested using a DNA fragmentation assay (Liu et al. (2000) Nucleic Acids Res. 28:4180-4188). It was found that administration of oral allimin induced the necrotic pathway for cell death of EAC cells as compared to control mice which received EAC but no allimin.

Therefore, the present invention is a novel polypeptide capable of controlling tumor cell growth, an anti-neoplastic effect. This polypeptide appears to be the only one identified to date that produces such effects in Ehrlich's ascitic carcinoma-induced malignancy in mice. Since nitric oxide is a potent tumoricidal agent (Farias-Eianer et al. (1994) Proc. Nati. Acad. Sci. 91:9407-9411), the stimulation of nitric oxide synthesis by allimin in both human and mouse erythrocytes is indicative of an anti-neoplastic effect of this protein. It has been found that oral administration of allimin was an effective anti-neoplastic agent, with a reduction in ascites fluid accumulation and an increase in the number of dead EAC cells in ascites fluid of treated mice.

Therefore, the present invention is a polypeptide that increases synthesis of nitric oxide in cells or tissues of the body and as a result is an effective treatment for cancer. In one embodiment, the polypeptide is allimin and it is administered to an animal, including humans, in a pharmaceutically acceptable carrier by a route that allows access to the cancerous tissue. For example, the polypeptide could be administered orally, by injection, or dermally. One of skill can appreciate that various compositions containing allimin could be prepared and formulated for administration to patients. Further, based on the teachings of the specification, one of skill would be able to determine an effective amount for increasing nitric oxide synthesis and for treating cancer in a patient.

The present invention is also a method for increasing nitric oxide synthesis in cells or tissues that involves contacting the cells or tissues with an effective concentration of allimin, wherein an effective concentration is an amount that has been shown to increase levels of nitric oxide in cells or tissues above the level that was present before contact with allimin. Other non-insulin polypeptides, similar to allimin, that have the ability to increase nitric oxide levels are also embraced by the present invention.

The present invention is a method for treating cancer by killing cancer cells. The method involves contacting cancer cells or tissues of an animal, including humans, with an effective amount of allimin, or other non-insulin polypeptide, so that nitric oxide levels in cells or tissues are increased in the cancer cells or tissues and then the cancer cells are killed. In the context of the present invention, an effective amount of allimin or the non-insulin polypeptide is an amount that is capable of increasing levels of nitric oxide in the cells or tissues above the levels that are present before treatment.

In a particular embodiment, the polypeptides of the present invention, both for use in treating cancer and as anti-diabetic agents, are administered orally. Polypeptides for oral administration are formulated with one or more pharmaceutically acceptable carriers (e.g., water, saline, etc.) and used in the form of ingestible tablets, buccal tablets, troches, capsules, elixirs, suspensions, syrups, wafers, chewing gums, foods and the like. Such formulations can be prepared by methods and contain carriers which are well-known in the art. A generally recognized compendium of such methods and ingredients is Remington: The Science and Practice of Pharmacy, Alfonso R. Gennaro, editor, 20th ed. Lippincott Williams & Wilkins: Philadelphia, Pa., 2000.

In general, such formulations and preparations contain at least 0.1% of active compound. The percentage of the compound and preparations can, of course, be varied and can conveniently be between about 0.1 to about 100% of the weight of a given unit dosage form. The amount of active agent in such compositions is such that an effective dosage level will be obtained.

Tablets, troches, pills, capsules, and the like can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring. The above listing is merely representative and one skilled in the art could envision other binders, excipients, and sweetening agents. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac or sugar and the like.

A syrup or elixir can contain the compositions of the present invention, sucrose or fructose as a sweetening agent, methyl and propylparabens as preservatives, a dye and flavoring such as cherry or orange flavor. Of course, any material used in preparing any unit dosage form should be substantially non-toxic in the amounts employed. In addition, the compositions of the present invention can be incorporated into sustained-release preparations and devices including, but not limited to, those relying on osmotic pressures to obtain a desired release profile.

A formulation of the present invention can be administered to a patient simultaneously with, or following, or both simultaneously with and following the administration of other therapeutic agents (e.g., a chemotherapy agent or a radionuclide) for enhancing the treatment of cancer.

Chemotherapy agents which can be used in combination with the compositions of the present invention include, cytotoxic agents such as TAXOL, Cytochalasin B, and Gramicidin D; antimetabolites such as Methotrexate, 6-Mercaptopurine, 6-Thioguanine, Cytarabine, 5-Fluorouracil, and Decarbazine; alkylating agents such as Mechlorethamine, Thiotepa, Chlorambucil, Melphalan, Carmustine (BCNU), Lomustine (CCNU), Cyclophosphamide, Busulfan, Dibromomannitol, Streptozotocin, Mitomycin C, Cis-Dichlorodiamine Platinum (II) (DDP), and Cisplatin; anthracyclines such as Daunorubicin (formerly Daunomycin) and Doxorubicin; antibiotics such as Dactinomycin (formerly Actinomycin), Bleomycin, Mithramycin, and Anthramycin (AMC); anti-mitotic agents such as Vincristine and Vinblastine; and other agents such as Selective Apoptotic Antineoplastic Drugs (SAANDs) such as APTOSYN® (Exisulind).

Radiation therapy agents can include external-beam radiotherapy, internal radioactive seed implants (Brachytherapy), and hemi-body radiation. Radiation therapy uses high-energy, ionizing radiation (e.g., gamma rays) to kill cancer cells. Ionizing radiation can be produced by a number of radioactive substances, such as Cobalt (Co-60), Radium (Ra-228), Palladium (Pd-103), Iodine (I-125), Radon (Rn-221), Cesium (Cs-137), Phosphorus (P-32), Gold (Au-198), Iridium (Ir-192), Boron (B-10), Actinium (Ac-225), Ruthenium (Ru-99), Samarium (Sm-153), and Yttrium (Y-90).

The pharmaceutical composition of the invention can further be administered in combination with agents which relieve side effects of cancer treatment. Such agents can be administered to a patient before, simultaneously with, or following, or before, simultaneously with and following the administration of the formulations disclosed herein. Examples of such agents which relieve side effects of cancer treatment include, Epoetin alfa to relieve symptoms of anemia; cell-protecting agents such as amifostine; and Strontium-89 and Samarium-153 for the relief of cancer-induced bone pain.

The present invention is further illustrated by the following non-limiting examples:

### EXAMPLE 1

### Purification of Allimin

Fifty grams of fresh garlic was obtained. The bulbs were collected and both the roots and the dried skin from the garlic were removed and thoroughly washed with distilled water to remove all adhering dirt and debris. The bulbs were homogenized to puree at 4° C. and the homogenate was immediately centrifuged at 7800×g at 4° C. The supernatant was collected and 50 ml of the supernatant and an equal volume of cold benzene (4° C.) was added in a separating funnel and thoroughly mixed by shaking for 15 minutes. The mixture was allowed to settle for 30 minutes at 4° C. After separation, 25 ml of aqueous phase was collected and again treated with an equal volume of cold benzene two more times. After the final benzene treatment, the aqueous phase was centrifuged at 31,000×g at 0° C. for 60 minutes. Five ml of the clarified aqueous extract was applied to a SEPHADEX® G 50 column (1×52 cm) pre-equilibrated with 10 mM sodium phosphate buffer pH 7.4 and the column was eluted with the same buffer with a flow rate of 1.5 ml/2 minute. Fractions (1.5 ml each) were collected and the ability of each fraction to stimulate nitric oxide synthesis from l-arginine in human erythrocytes was determined. Fractions containing the highest activity (#86-#93) emerged from the column in a single peak. The fractions were pooled (12 ml) and dialyzed against 0.9% NaCl at 4° C. overnight and stored at −20° C. for further studies. The dialyzed material was found to be a homogeneous preparation of protein which is referred to as allimin.

### EXAMPLE 2

### Nitric Oxide Synthesis in Erythrocytes

The stimulation of nitric oxide synthesis was determined by assaying 0.1 ml of aqueous extracts of garlic or benzene purified allimin or different fractions from the SEPHADEX® column to erythrocyte suspensions in Kreb's buffer, pH 7.4. The formation of nitric oxide in the reaction mixture was quantitated by the conversion of oxyhemoglobin to methemoglobin. Quantitative amounts of nitric oxide in the reaction mixture were confirmed independently by chemiluminescence technique. Briefly 1.0 ml of the reaction supernatant was treated with 15 mM (final concentration) of oxyhemoglobin under N₂. The spectral changes at 650, 630 and 575 nm due to the conversion of oxyhemoglobin to methemoglobin was continuously monitored by using a scanning BECKMAN spectrophotometer to quantitate nitric oxide formation using greater than 99% percent pure preparation of nitric oxide.

### EXAMPLE 3

### Preparation of Erythrocyte Suspensions

Blood was collected from healthy male and female volunteers, between the ages of 25-50 years, who had not taken any medication for at least for 14 days prior to the donation of blood. The blood was anti-coagulated by adding 1 volume 130 mM sodium citrate to 9 volumes of blood, mixed by gentle inversion, and the erythrocyte suspension was prepared.

For the preparation of erythrocyte suspensions from mice, blood was drawn from the tail vein and anti-coagulated by adding sodium citrate to the blood sample and a suspension was made as described by Ray et al. ((1986) Biochim. Biophys. Acta 856:421-427).

### EXAMPLE 4

### Polyacrylamide Gel Electrophoresis

The homogeneity of purified allimin was determined by SDS-polyacrylamide gel electrophoresis under alkaline conditions. The molecular weight and subunit composition of the purified protein was determined by SDS-polyacrylamide (15%) gel electrophoresis of reduced (using 0.1 M dithiothreitol) and non-reduced allimin. The gels were stained with 0.02% COOMASSIE® brilliant blue.

### EXAMPLE 5

### Matrix-assisted Laser Desorption

The molecular weight of the purified protein was further ascertained by mass spectrometry. Amino acid sequence analysis of allimin was performed on a commercially available analysis unit (i.e., Perspective Biosystem Voyager DESTR). The amino acid sequence was determined by using the PE/ABD HT protein sequencing system.

### EXAMPLE 6

### Animals

Inbred albino mice of both sexes were raised in an institutional facility from birth to age 2 months and weighed between 20-25 grams. These mice were allowed free access to feed and sterile water under 12 hour cycles of alternating light and dark. A total of 60 mice were studied. Group I consisted of 20 mice that were divided into 4 subgroups of 5 mice per subgroup. Various tissue samples were taken from the mice and prepared to compare the effects of insulin and allimin. Group II consisted of 20 mice that were divided in 4 groups, 5 mice in each subgroup. All of the mice were made diabetic, one of the diabetic subgroup of mice received insulin and the second diabetic subgroup received oral administration of allimin. The plasma nitric oxide levels in the Group II mice were measured.

### EXAMPLE 7

### Diabetic Mice and Blood Glucose Determination

Alloxan was used to induce diabetes in mice in accordance with standard methods. Blood glucose levels were determined by using glucose oxidase strips in a glucometer. The mice were considered to be diabetic when the blood glucose level after overnight fasting were 250-300 mg/dl as compared to 70-80 mg/dl in non-diabetic mice. These diabetic mice were determined to have <1.0 μunit of insulin/ml of plasma as determined by radioimmunoassay. When these diabetic mice were fed ad libitum, the blood glucose level increased to 400-450 mg/dl after 2 hours of feeding.

### EXAMPLE 8

### Membrane Tyrosine Kinase Activity

The effect of allimin on the activation of epitrochlearis muscle membrane was determined using monoclonal anti-phosphotyrosine kinase antibody, Glu⁸⁰ Tyr⁸⁰ copolymer, and [³²P]-ATP and compared with that of insulin.

### EXAMPLE 9

### Assay of PI₃ Kinase

The activation of PI₃ kinase of mice epitrochlearis muscle membrane either by allimin or insulin was determined using PI₃ kinase antibody. The membrane was prepared and solubilized in 0.01% TRITON™ X100 and immunoprecipitated by PI₃ kinase antibody and protein A SEPHAROSE®. The precipitate was treated with either allimin or insulin in the presence of phosphatidyl inositol and [³²P]-ATP. The PI₃ kinase activity was determined by CHCl₃—CH₃OH extraction and HPLC.

### EXAMPLE 10

### Determination of Glucose Oxidation

The effect of allimin on the oxidation of glucose was compared with that of insulin. Typically, mice epitrochlearis muscle was incubated with 6 mM glucose containing 10 μCi of [U-¹⁴C]-D-glucose in 1.5 ml of Krebs-Ringer buffer, pH 7.4, in the presence of different concentrations of allimin or insulin. Glucose oxidation was determined.

### EXAMPLE 11

### Glucose Transport Activity

Mice epitochlearis muscle and the membrane of the muscle were prepared. Glucose transport activity was determined by incubating 0.4 to 0.7 grams of epitochlearis muscle in Krebs-Ringer buffer, pH 7.4, in the presence of either insulin (200 units/ml) or allimin (50 g/ml) using a 2-deoxy D-¹⁴C glucose. Glucose oxidation was determined by incubating 1.0 to 1.2 grams of epitrochlearis muscle in Krebs-Ringer buffer, pH 7.4, in the presence of either insulin or allimin as indicated using U-14C glucose. Insulin- or allimin-induced activation of tyrosine kinase and PI₃ kinase activities of the membrane preparation were determined using anti-phosphotyrosine antibody and PI₃ kinase antibody, respectively, as described. Results shown in Table 4 are mean ±SD of 5 to 6 experiments using different animals.

### EXAMPLE 12

### Platelet Aggregation

Given the role of nitric oxide in the function of platelets it is was determined whether allimin could modulate platelet aggregation. Platelet Rich Plasma (PRP) was isolated from citrated blood and incubated with 40 nm Allimin for 30 minutes at 23° C. Subsequently, 6 μM adenosine 5′-diphosphate (ADP) was added and inhibition of ADP-induced platelet aggregation was recorded and compared with a control. The results of this analysis demonstrated that Allimin was a potent inhibitor of platelet aggregation at 40 nM concentration of Allimin and 6 μM ADP concentrations.

What is claimed:

1. A isolated non-insulin polypeptide for increasing nitric oxide synthesis in cells or tissues, wherein said polypeptide comprises the amino acid sequence Glu-Gly-Leu-Tyr-Ala-Gly-Gln-Ser-Leu (SEQ ID NO:15).

2. A pharmaceutical composition comprising the non-insulin polypeptide of claim 1 in admixture with a pharmaceutically acceptable carrier.

3. A method for increasing the synthesis of nitric oxide in cells or tissues comprising contacting a cell surface insulin receptor with the non-insulin polypeptide of claim 1.

4. A method for preventing or controlling hyperglycemia comprising administering to an animal in need of treatment an effective amount of a pharmaceutical composition of claim 2 thereby preventing or controlling hyperglycemia in the animal.

5. The method of claim 4, wherein the pharmaceutical composition is administered orally.

6. A method of increasing insulin production in an animal comprising administering to an animal in need of treatment an effective amount of a pharmaceutical composition of claim 2 thereby increasing insulin production in the animal.

7. The method of claim 6, wherein the pharmaceutical composition is administered orally.

8. A method for killing cancer cells in an animal comprising administering to an animal in need of treatment an effective amount of a pharmaceutical composition of claim 2 thereby increasing nitric oxide synthesis in said cancer cells and killing said cancer cells.

9. The method of claim 8, wherein the pharmaceutical composition is administered orally.


SEQUENCE LISTING (8 sequences)

SEQ ID NO: 1 - protein, 35 aa, Allium sp.
  MISC_FEATURE (1)..(1): Xaa represents an unidentified amino acid.
  MISC_FEATURE (33)..(33): Xaa represents an unidentified amino acid.
  MISC_FEATURE (35)..(35): Xaa represents an unidentified amino acid.
XMIPTNGEGLYAGQSLDVEQYKFIMRPDDNLVXYX

SEQ ID NO: 2 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 3 - protein, 7 aa, Homo sapiens
EDLQVGQ

SEQ ID NO: 4 - protein, 7 aa, Allium sp.
EGLYAGQ

SEQ ID NO: 5 - protein, 16 aa, Homo sapiens
PLALEGSLQKRGIVEQ

SEQ ID NO: 6 - protein, 17 aa, Allium sp.
PTNGEGLYAGQSLDVEQ

SEQ ID NO: 7 - protein, 15 aa, Homo sapiens
GGGPGAGSLQPLALE

SEQ ID NO: 8 - protein, 13 aa, Allium sp.
GEGLYAGQSLDVE